(19) United States
(12) Patent Application Publication
(10) Pub. No.: US 2006/0276838 A1
(43) Pub. Date: Dec. 7, 2006

(54) Vascular puncture sealing method, apparatus, and system

(76) Inventors: Jeffrey P. Wensel, Eugene, OR (US); Patrick J. Bergin, Eugene, OR (US)

Correspondence Address: STATTLER, JOHANSEN, AND ADELI LLP, 1875 CENTURY PARK EAST SUITE 1360, LOS ANGELES, CA, 90067, US

(21) Appl. No.: 11/354,225

(22) Filed: Feb. 13, 2006

Related U.S. Application Data
(60) Provisional application No. 60/688,510, filed on Jun. 7, 2005.
(60) Provisional application No. 60/693,706, filed on Jun. 24, 2005.
(60) Provisional application No. 60/723,878, filed on Oct. 5, 2005.

(51) Int. Cl. A61B 17/08

(52) U.S. Cl. 606/213

(57) ABSTRACT

Some embodiments of the invention provide a guidewire directed vascular sealing device for securing hemostasis within a vascular puncture and puncture tract extending from the epidermis into a blood vessel in a living being. In some instances, the puncture tract is created by a percutaneous access apparatus used in the performance of medical catheter based diagnostic and therapeutic techniques. In some embodiments, the apparatus includes a hemostatic compound or material designed to facilitate clot formation within the tract.

### REFERENCE TO RELATED APPLICATIONS

This application claims priority to (1) U.S. Provisional Application filed on Jun. 7, 2005, assigned Ser. No. 60/688,510 and titled “Hemostatic Wire Guided Bandage”, (2) United States Provisional Application filed on Jun. 24, 2005, assigned Ser. No. 60/693,706 and titled “Vascular Puncture Sealing Device and Method of Use,” and (3) United States Provisional Application filed on Oct. 5, 2005, assigned Ser. No. 60/723,878 and titled “Vascular Puncture Sealing Mechanism and Method of Use.” All three of the applications are incorporated herein by reference.

### FIELD OF THE INVENTION

The invention pertains to vascular puncture sealing mechanisms.

### BACKGROUND

Numerous medical diagnostic and therapeutic procedures require access to the internal organs of an organism. Some of these procedures can be performed without traditional surgical incisions by utilizing catheter-based mechanisms to enter blood vessels. Usually, catheter-based mechanisms require a needle inserted through the skin and directed into a blood vessel. This creates a conduit for extending a guide wire through the needle and into the blood vessel. After positioning the guide wire, the needle can be removed and a hollow tube or catheter directed over the guide wire into the blood vessel. The tube or catheter provides access for administration of certain substances and/or for passage of additional equipment that will be subsequently used to perform manipulations within the vasculature or within other organ systems accessible through the vasculature.

To prevent bleeding upon completion of a catheter-based intravascular procedure, the catheter must be removed and the puncture quickly sealed. In the low-pressure environment of the venous system, a small needle puncture is readily sealed by the brief application of pressure to the site and application of a light dressing, such as a bandage. This method is widely utilized after needle stick procedures such as blood drawings.

However, when punctures are created with larger caliber mechanisms, such as catheters, in the high-pressure environment of arteries, the puncture created will not readily seal with the application of brief pressure. Prolonged external pressure lasting ten to twenty minutes is not uncommon. Such pressure may lead to substantial patient discomfort at the puncture site and/or a significant failure rate with late bleeding and hematoma formation.

In the past, several methods have been proposed to address this problem. For instance, one prior apparatus utilizes a marker to indicate the position of the bandage with respect to the wound to be treated in order to position externally applied pressure at or near a puncture site. Another apparatus uses a pad which, when moistened by fluid from a wound, expands and exerts pressure against a wound.

Another apparatus utilizes laser energy directed through a balloon tipped catheter into the vascular tract and positioned just outside the outer wall of the blood vessel. The balloon is used to create a covering for the vascular puncture. The laser is used to create a laser “weld” or seal in the adjacent tissue.

Another apparatus uses both a balloon tipped catheter and an absorbable plug. The plug is used to occlude the vascular access tract and provide hemostasis. The balloon tipped catheter serves as a positioning anchor for antegrade insertion of the vascular plug and must be removed from the patient after plug deployment.

Yet another apparatus uses a balloon tipped catheter arranged so as to pass into the vascular lumen by means of the extant access sheath. After this procedure it is withdrawn to the intraluminal side of the blood vessel puncture to provide temporary hemostasis. A pro-coagulant slurry is then injected into the vascular access tract to promote coagulation. During this time, the balloon tipped catheter remains inflated. After a suitable period of time necessary to promote blood coagulation, the balloon tipped catheter is deflated and withdrawn from the access tract.

Each of these approaches has its own unique set of shortcomings. The prior mechanisms require either (1) leaving a component within the vascular lumen, or (2) withdrawing the component. Leaving a component within the vascular lumen creates the possibility that the component may become disengaged prior to absorbing into the body, thus potentially leading to blood clots. Withdrawing the component creates an additional channel through which blood may flow, thereby jeopardizing hemostasis.

Therefore, there is a need in the art for a mechanism that achieves hemostatic closure of a vascular puncture site without leaving a component within the vascular lumen or requiring the withdrawal of the component. Ideally, such a mechanism would quickly, painlessly and reliably achieve hemostasis upon withdrawal of vascular catheters and/or other such mechanisms, and consequently reduce patient discomfort, staff time and failure rate associated with vascular hemostasis.

### SUMMARY

Some embodiments of the invention provide a system for achieving hemostasis in a wound after a medical procedure that creates a puncture tract. In such procedures, the puncture tract is also called an access tract. The access tract is usually created by a needle used in the performance of medical catheter based diagnostic and therapeutic techniques and normally extends from the epidermis to the vasculature in a living organism.

The system of some embodiments includes (1) a measuring mechanism for measuring the distance from the skin to a vascular puncture, (2) a sealing mechanism for placing a hemostatic plug at the vascular puncture to occlude the puncture, and (3) a clamp for stabilizing the sealing mechanism during the recovery period and preventing the plug from moving from the vascular puncture.

In some embodiments, the measuring mechanism measures the distance from the skin to a vascular puncture. Knowing this distance allows the precise placement of the plug within the vascular tract. In some embodiments, the occlusive plug affixes to a tubular member that is inserted over a guidewire and into a vascular puncture tract. The tubular member of the sealing mechanism advances the plug over the guidewire and into the access tract. By using the guidewire, the plug is accurately centered at the vascular puncture and the surrounding vascular wall with the tip of the plug intruding into and occluding vascular puncture. In some embodiments, the plug, or portion of it (e.g., the plug's tip), is coated with, contains, or is completely composed of a pro-coagulant material such as Chitosan to facilitate hemostasis. In some embodiments, the plug is made from bioabsorbable material that allows the plug to dissolve harmlessly into the organism over time.

### BRIEF DESCRIPTION OF THE DRAWINGS

The novel features of the invention are set forth in the appended claims. However, for purpose of explanation, several embodiments of the invention are set forth in the following Figures.

FIG. 1 illustrates a hemostasis access sheath placed over a flexible guidewire and advanced through a percutaneous opening, an epidermal layer, and a subcutaneous layer into a blood vessel, creating an access tract.

FIG. 2 illustrates the same flexible guidewire and access tract after removal of the hemostasis sheath and completion of a vascular puncture sealing procedure.

FIG. 3 illustrates a measuring device with markings that measures the distance between the percutaneous opening and the vascular puncture.

FIG. 4 illustrates the measuring device without markings that measures the distance between the percutaneous opening and the vascular puncture.

FIG. 5A illustrates a sealing instrument which includes one or more support tubes and a tapered-tip plug, where the opposing end of an inner support tube in some embodiments, extends beyond an outer support tube and connects to a disc-shaped surface.

FIG. 5B illustrates the distal end of both of the inner and outer support tubes housed within a cup formed in the plug.

FIG. 5C illustrates the exterior surface of the sealing instrument of some embodiments where markings are used to position the plug at the vascular puncture site.

FIG. 5D illustrates the cup of the plug including a central lumen, which defines the central passageway through the tip and the cup section of the plug.

FIG. 5E illustrates some of the other possible shapes of the plug.

FIG. 6 illustrates the insertion of the guidewire into the central lumen until the plug's tip is positioned at the vascular puncture.

FIG. 7 illustrates the positioning of the plug at the vascular puncture site.

FIG. 8 is a perspective of a clamping mechanism of some embodiments of the invention.

FIG. 9 is a top elevation of the clamping mechanism of some embodiments of the invention.

FIG. 10A illustrates a partially closed position defined by a position of a protrusion through a central lumen that is defined within a rigid arm of the clamping mechanism.

FIG. 10B illustrates a fully closed position defined by a position of the protrusion through the central lumen that is defined within the rigid arm of the clamping mechanism.

FIG. 11 illustrates the clamping mechanism used in some embodiments of the invention to secure the sealing mechanism in position.

FIG. 12 illustrates the plug placement and vascular sealing mechanism.

FIG. 13 illustrates the plug placement after withdrawal of the guidewire.

FIG. 14 illustrates that in some embodiments, the wall of the outer support tube flares inwardly such that its exterior circumference is smaller at the proximal end of the tube and larger at its opposing end after the inner support tube is inserted into the outer support tube.

FIG. 15 illustrates the varying thickness of the outer support tube and the distal end of each tube resting firmly within and cooperating with the cup of the plug.

FIG. 16 illustrates the withdrawal of the inner support tube, thereby allowing the circumference of the distal end of the outer support tube to return to its normal and more contracted position.

FIG. 17 illustrates the vascular sealing mechanism secured with the clamp mechanism in place.

FIG. 18 illustrates the outer support tube and remaining apparatus removed, causing the access tract to close naturally and facilitate hemostatis within the access tract.

FIG. 19 illustrates the access tract as being roughly perpendicular to the plane of the vascular puncture in some embodiments.

FIG. 20 illustrates another plug and sealing mechanism of some embodiments of the invention.

FIG. 21 illustrates the plug having a proximal end that is detachably affixed to the distal end of the sealing mechanism.

FIG. 22 illustrates the plug having prongs that extend beyond the cup.

FIG. 23 illustrates the distal end of the outer support tube as including a flared section that extends radially outward at an angle, where the flared section includes prongs and a receptacle placed between each pair of prongs.

FIG. 24A illustrates a plug of other embodiments of the invention where the prongs arch radially outwards at all times and are more flexible.

FIG. 24B illustrates the plug including a tapered tip and prong ends.

FIG. 24C illustrates the plug having a shoulder.

### DETAILED DESCRIPTION OF THE INVENTION

In the following description, numerous details are set forth to provide a better understanding of the various embodiments of the invention. However, one of reasonable skill in the art will realize that the invention may be practiced without the use of the specific details presented herein. In some instances of describing the invention, well-known structures and apparatus may be shown in block diagram form to avoid obscuring the description of the invention with unnecessary detail. Therefore, the examples provided herein for clarification and understanding should not be read into and thereby limit the language of the claims.

Some embodiments of the invention provide an apparatus for achieving hemostasis after a medical procedure that creates a puncture tract in a living organism. To better understand these embodiments, it is helpful to understand relevant terminology and at least one environment in which the apparatus is used. Therefore, Section I presents relevant terminology, while Section II provides an overview of intravascular procedures (which provide a relevant environment in which the apparatus is used). Finally, Section III presents a vascular puncture sealing device.

I. Definitions and Terminology

An opening in the skin is called a percutaneous opening because it passes through the skin. The subcutaneous layer is the layer immediately below the skin, which is composed of the epidermal and dermal layers. The hole from the percutaneous opening to the blood vessel is the puncture tract or access tract. The opening in the blood vessel wall is a vascular puncture or vascular opening. The open space within the blood vessel is called the vascular lumen. As used in the following discussion, a “lumen” is an opening, such as the cavity of a tubular organ or the bore of a tube (as of a hollow needle or catheter).

Having identified some of the relevant terms used herein, an exemplary intravascular procedure is now described.

II. An Exemplary Intravascular Procedure

Some embodiments of the invention have particular utility when utilized in conjunction with intravascular procedures commonly performed by radiologists and cardiologists. Examples of such procedures include angiography, angioplasty, vascular stenting and stent graft placement, arterial thrombectomy, arterial embolization, intra-arterial drug administration, etc. These procedures normally involve the insertion of a hollow needle (e.g., an 18 gauge thin walled needle) through the skin. The needle advances through the body tissue overlying a blood vessel and continues through the proximal side of the vascular wall until the distal tip of the needle enters the vascular lumen. A brisk return of blood through the needle hub signals entry of the needle into the vascular lumen.

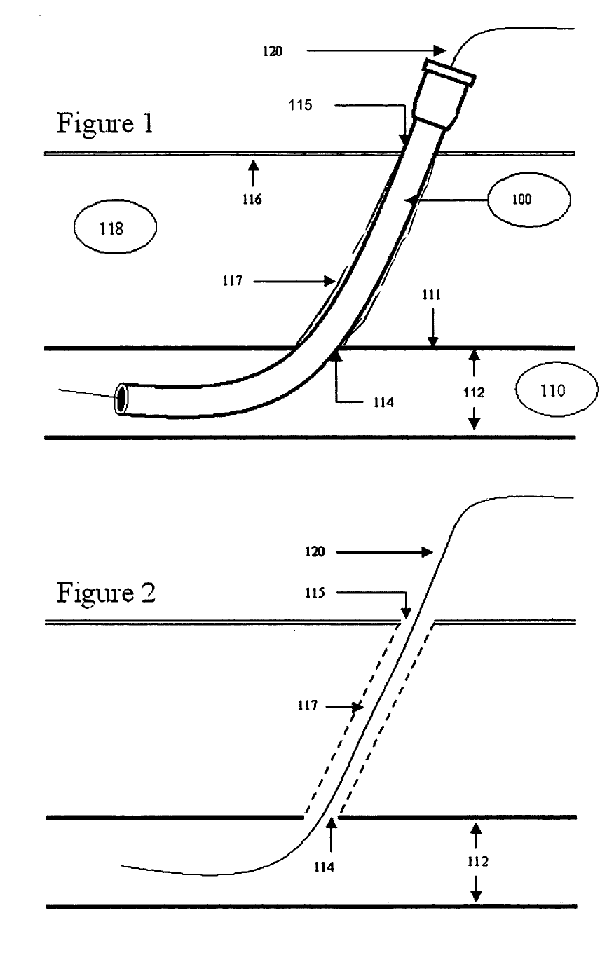

FIGS. 1 and 2 illustrate an exemplary intravascular procedure that commonly uses an access sheath 100 placed in the access tract 117 to facilitate entry into the vascular lumen 112 by diagnostic and therapeutic tools. FIG. 1 illustrates a hemostasis access sheath 100 placed over a flexible guidewire 120 and advanced through a percutaneous opening 115, the epidermal layer 116, and a subcutaneous layer 118 into a blood vessel 110, creating an access tract 117. Also shown are a vascular puncture 114, a vascular wall 111 and a vascular lumen 112. In this configuration, the site has been prepared for an intravascular procedure, which may be in process or may have completed.

To install the access sheath 100, the operator first creates an access path to the blood vessel 110 by cutting a percutaneous opening 115 in the epidermal layer 116 at a point that is favorable to accessing the blood vessel 110. A needle or other cutting tool is typically advanced through a percutaneous opening 115, an epidermal layer 116, a subcutaneous layer 118 and a vascular wall 111. It continues through the vascular wall 111 (creating a vascular puncture 114) and into a vascular lumen 112 of a blood vessel 110. This creates the access tract 117.

After creating the access tract 117, the operator must thread a guidewire 120 longitudinally through the needle previously referenced. The guidewire can be made of any flexible material, such as a metal, metal alloy or synthetic polymer. After positioning the guidewire 120 within the access tract 117, the needle may be removed while maintaining the guidewire 120 in position. Normally, an access sheath 100 is later placed within the access tract 117 to prevent the tract 117 from closing during the procedure. The access sheath 100 is therefore threaded onto the guidewire 120 and inserted into the access tract 117, using the guidewire 120 to position the sheath 100 precisely into place. When positioned at its final location, one end of the sheath 100 is within the vascular lumen 112 while the opposing end is outside of the organism. Once the access sheath 100 is in place, other apparatus and/or materials can pass through the access sheath 100 and advance into the blood vessel 110 to the area of interest within the body.

Upon completion of the intravascular procedure, the catheters and other apparatus used in the procedure are removed from the blood vessel 110. This is generally followed by the removal of the sheath 100 over the guidewire 120, leaving the guidewire 120 in place within the access tract 117 and leaving the access tract 117 open. FIG. 2 shows the same flexible guidewire 120 and access tract 117 after removal of the hemostasis sheath and completion of the procedure.

If hemostasis is not quickly attained after removal of the sheath 100 from the access tract 117, vigorous bleeding can occur. Therefore, the vascular puncture 114 and the access tract 117 must be sealed as quickly and as efficiently as possible. One method of doing so uses a hemostatic wire guided vascular puncture sealing mechanism.

III. A Vascular Puncture Sealing Mechanism

Some embodiments of the invention provide an apparatus for achieving hemostasis in a wound after a medical procedure that creates a puncture tract. In some embodiments, the apparatus includes a plug that is inserted in a vascular puncture to achieve hemostasis. The apparatus in some embodiments also include a mechanism for delivering the plug into the puncture tract. In some embodiments, the mechanism not only positions the plug, but also occludes the opening of the puncture tract and the vascular puncture. Although some embodiments of a hemostatic wire guided vascular puncture sealing mechanism achieve hemostasis at a vascular puncture site in a living organism, the apparatus' construction and use has widespread applicability in analogous non-vascular settings.

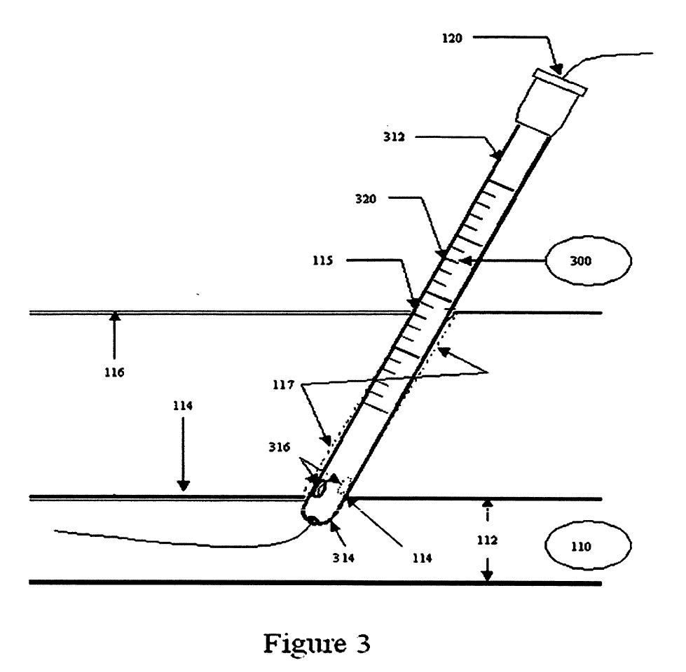

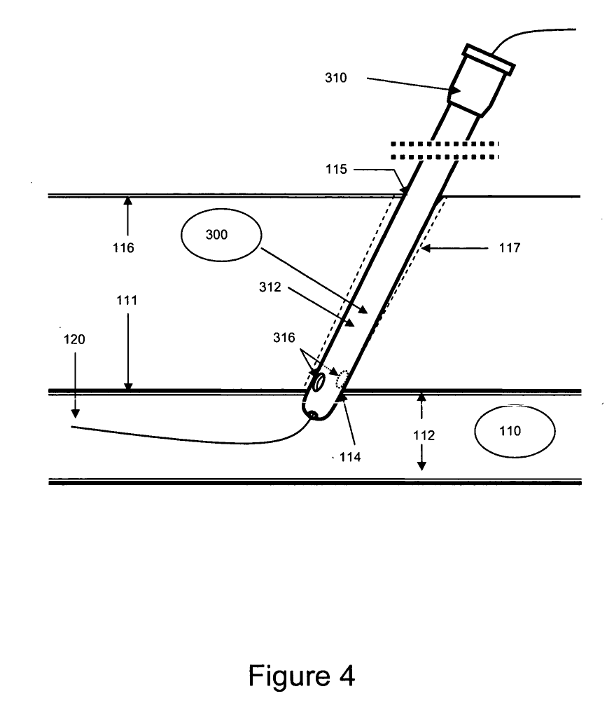

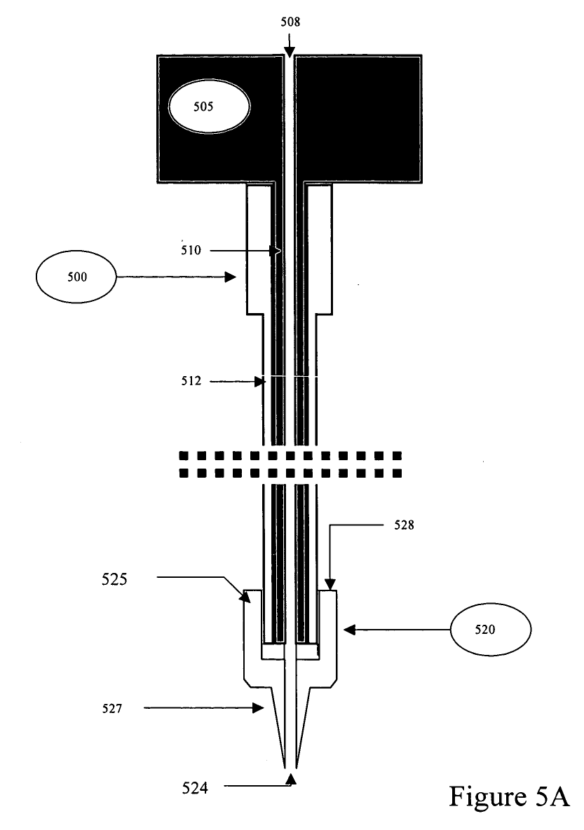

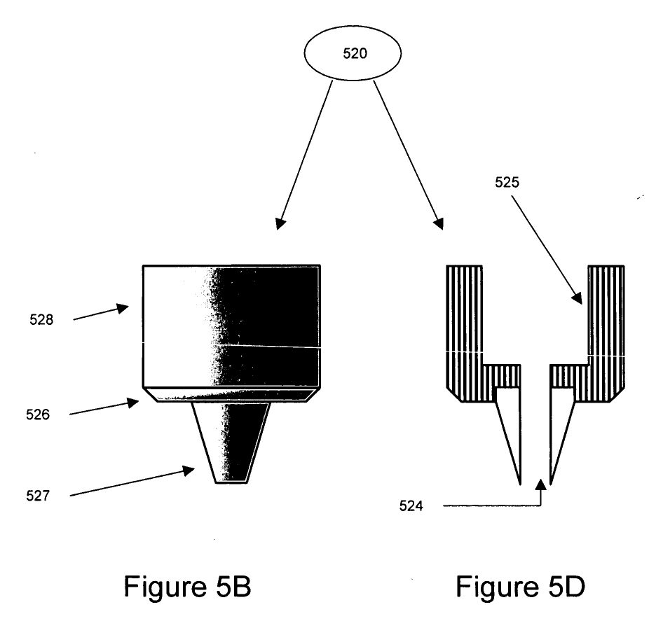

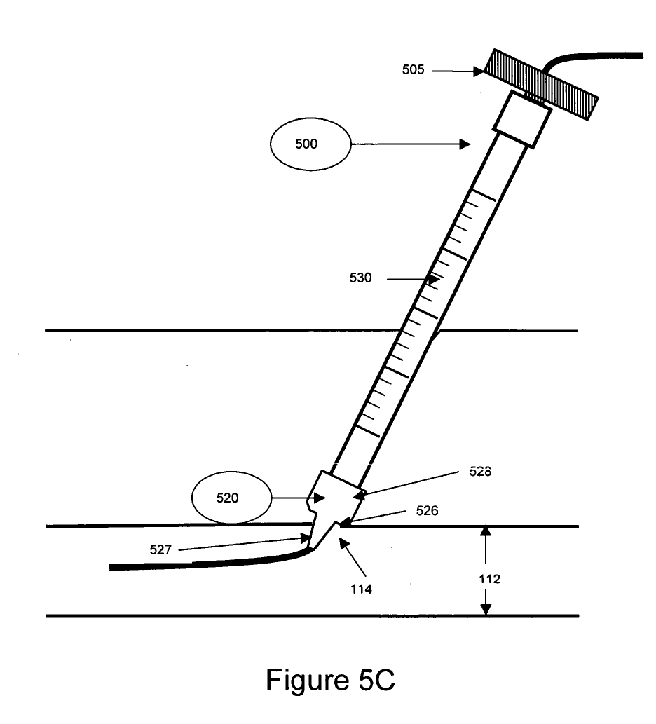

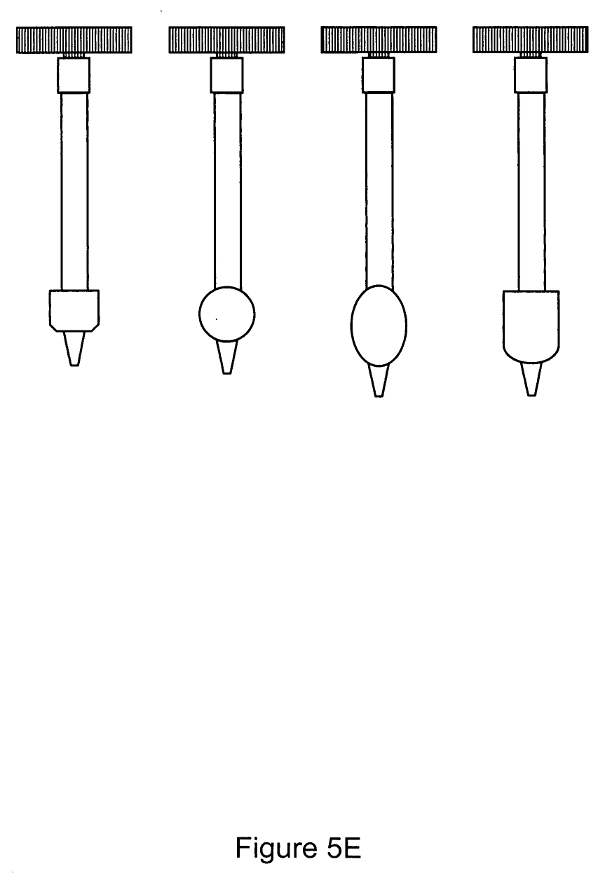

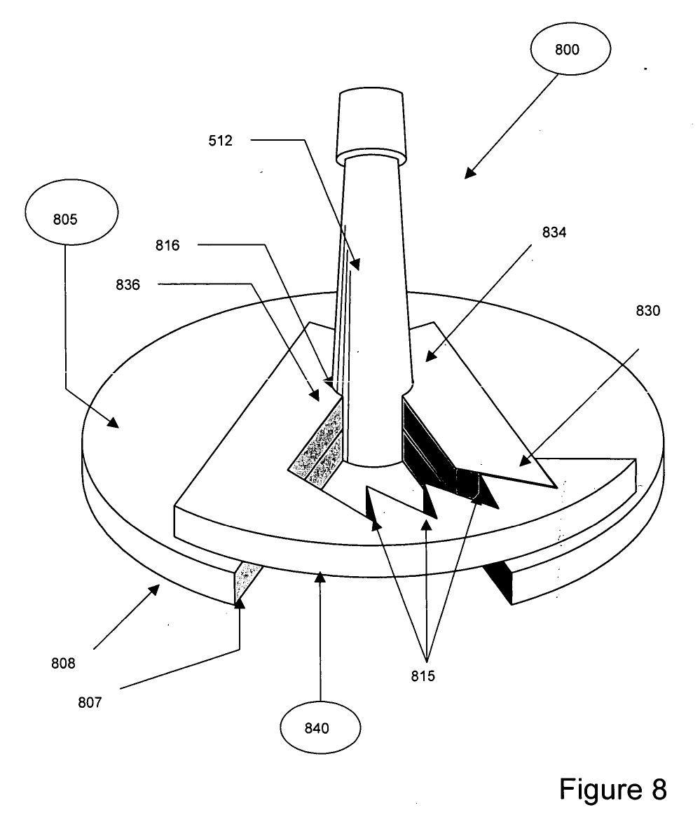

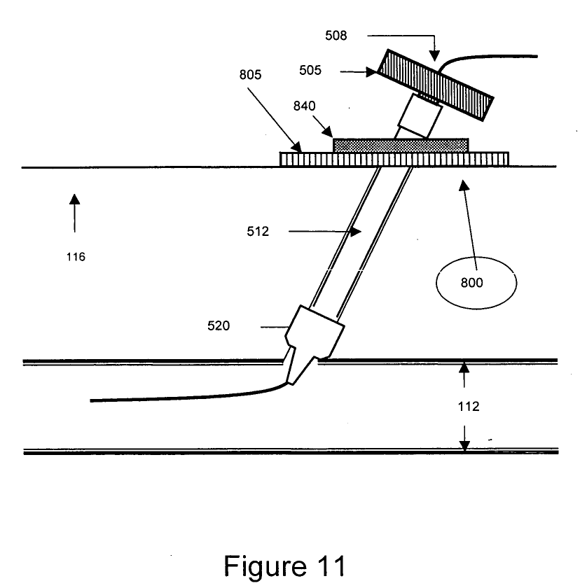

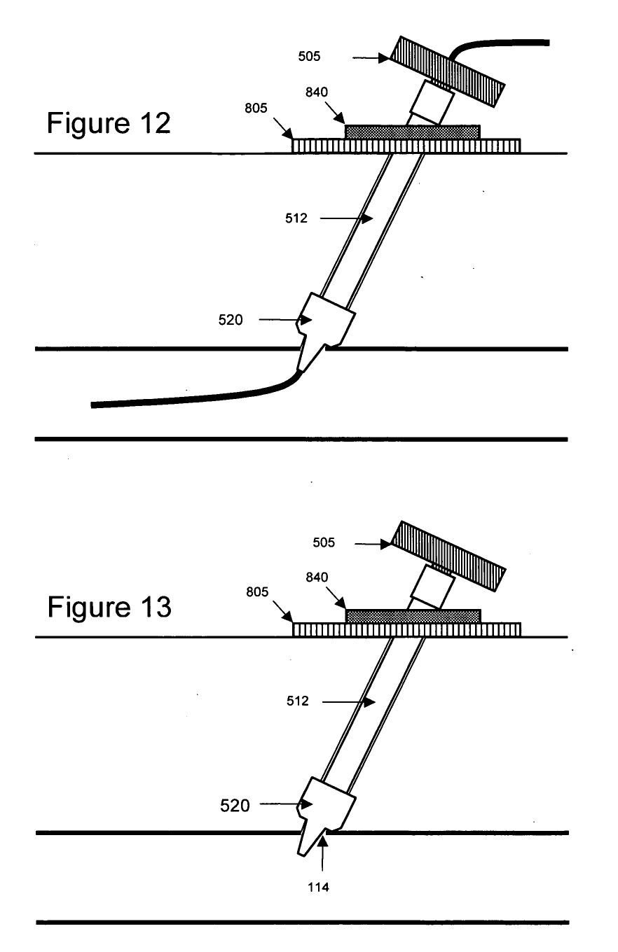

FIGS. 3 through 16 illustrate various components of a mechanism and system to seal a vascular puncture 114. FIGS. 3 and 4 illustrate a measuring device that measures the distance between the percutaneous opening 115 and the vascular puncture 114. FIGS. 5 through 7 and 12 through 16 illustrate a plug placement and vascular sealing mechanism. FIGS. 8 through 11 illustrate a clamping mechanism.

A. The Component Parts of a Vascular Puncture Sealing Mechanism

1. The Measuring Instrument

The measuring instrument of the vascular puncture sealing mechanism precisely measures the distance from the percutaneous opening 115 to the vascular puncture 114. FIGS. 3 and 4 depict the measuring apparatus 300 of some embodiments of the measuring mechanism 300 as it is advanced into the vascular lumen 112 and then withdrawn from the vascular lumen 112.

As seen in these figures, the measuring mechanism 300 includes a hollow tube 312, an opening at each end, and side holes 316 near one end. The tube's 312 outside surface has markings 320 spaced at predetermined (such as 1 mm) intervals. However, the indicators used in the measuring mechanism 300 are not limited to a specific type of mark. Any indicator that is capable of observation can be used. One end of the tube 312 has a rounded tip 314 with an opening large enough to allow the passage of the guidewire 120 through it. In some embodiments, the measuring mechanism 300 may also include an alignment tab to assist with the proper alignment of the holes 316 with respect to the blood vessel 110.

The side holes 316 allow blood to enter the measuring mechanism 300 through the holes 316 and exit through the opposite end. Therefore, the holes 316 are at a defined angle with respect to each other on the tube 312 so that when the tube 312 is inserted at an angle into or withdrawn from the vascular lumen 112, the holes 316 enter or exit the blood vessel 110 simultaneously. In other words, any angle for entry of the measuring device 300 into the vascular lumen 112 requires the side holes 316 to be located on opposing sides of the tube 312 with one hole positioned slightly more distally beyond the other.

To measure the distance from the percutaneous opening 115 to the proximal exterior surface of the blood vessel wall 111, the operator advances the measuring mechanism 300 over the guide wire 120 and into the vascular lumen 112. Entry into the vascular lumen 112 is signaled by brisk blood return at the proximal hub 310 of the measuring mechanism 300.

After the distal tip 314 of the measuring mechanism 300 has entered the vascular lumen 112, the measuring mechanism 300 is slowly withdrawn from the vascular lumen 112. The withdrawal continues until the holes 316 exit from the vascular lumen 112 (as shown in FIG. 4) and enter the access tract 117. This entry is signaled by an abrupt drop in amount of blood at the hub 310. At this point, the operator may use the measurement indicators placed on the outside of the measuring mechanism 300 to determine the distance from the percutaneous opening 115 to the vascular puncture 114.

After the measurements have been completed and recorded, the vascular puncture 114 must be quickly sealed with a vascular puncture sealing instrument 500 to prevent undue bleeding.

2. The Vascular Puncture Sealing Instrument

The sealing instrument 500 positions a plug 520 at the distance measured by the measuring mechanism 300. In other words, this instrument positions the plug 520 at the vascular puncture site 114 to occlude the vascular puncture 114. FIGS. 5 through 7 illustrate various details of the sealing instrument 500 of some embodiments. As shown in FIGS. 5A and 7, the instrument 500 in some embodiments includes one or more support tubes 510 and 512 and a tapered-tip plug 520. The support tubes, 510 and 512, position the plug 520 to occlude the vascular puncture 114 (illustrated in FIG. 5C). Upon positioning the plug 520, it intrudes into, occludes and thereby seals the vascular puncture 114.

a) The Support Tubes

The support tubes form the primary components of a delivery mechanism used to position a plug 520 to occlude the vascular puncture 114. Referring now to FIG. 5A, the support tubes 510 and 512 include an outer support tube 512 and an inner support tube 510. In some embodiments, the distal end of both of these tubes 510 and 512 is housed within a cup 528 formed in the plug 520, as shown in FIGS. 5B and 14-16.

Referring still to FIG. 5A, the exterior surface of the outer support tube 512 also forms the exterior surface of the instrument 500. As shown in FIG. 5C, the exterior surface has markings 530 (e.g., equidistant hash marks of the same measure as those on the measuring instrument 300) in some embodiments that are used to position the plug 520 at the vascular puncture site 114, as further described below.

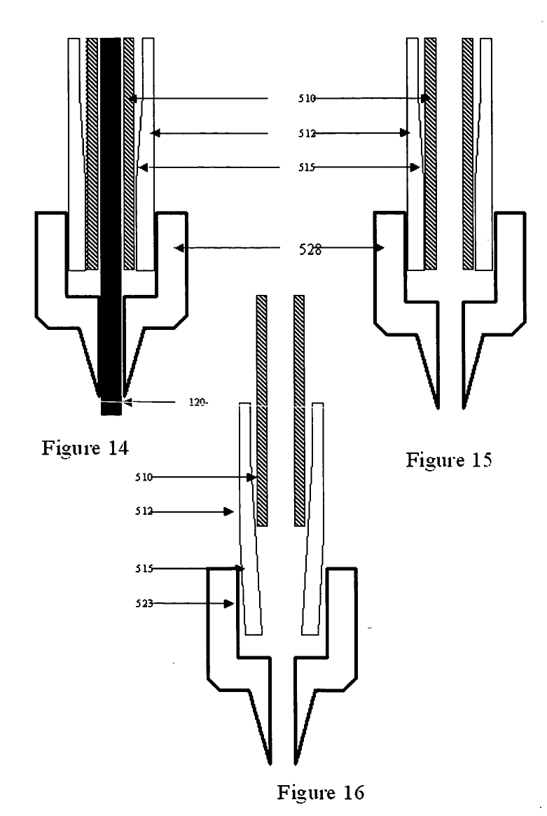

The outer support tube 512 assists in the delivery of the plug 520 to the vascular puncture site 114. As illustrated in FIGS. 14-16, the thickness 515 of the outer support tube 512 varies (as indicated by marked annotation 515 in FIGS. 14-16) for reasons detailed below. In some embodiments, the outer support tube wall contains longitudinal slits. When lateral pressure is applied, these slits allow the circumference of the outer support tube 512 to expand radially outward from the center of the tube 512. In other embodiments, the wall of the outer support tube 512 flares inwardly (FIGS. 14-16) such that its exterior circumference is smaller at the proximal end of the tube 512 and larger at its opposing end after the inner support tube 510 is inserted into the outer support tube 512 (FIG. 14-15). The outer support tube 512 is made from semi-flexible material such as deformable plastic, polymer, or similar deformable or compliant material that is flexible enough to expand radially outward when pressure is applied from the center of the tube 512.

When a device, such as the inner support tube 510, is inserted into the distal end of the outer support tube 512, the outer tube's circumference expands at the distal end. With the appropriately sized inner tube 510, the expansion of the distal exterior circumference of the outer support tube 512 continues until the exterior circumference comes into close contact with the interior surface of the cup 528 of the plug 520. The lateral pressure of the outer support tube 512 wall against the interior surface of the cup 528 is sufficient to allow the cup 528 to affix to and cooperate with the outer support tube 512 while the plug 520 is being positioned within the access tract 117. When the plug 520 is positioned, the inner support tube 510 may be withdrawn. As shown in FIG. 16, the withdrawal of the inner support tube 510 allows the circumference of the distal end of the outer support tube 512 to return to its normal and more contracted position. This contraction naturally pulls the tube's circumference away from the interior surface of the cup 528. With neither of the tubes 510 and 512 cooperating with the cup 528, the tubes 510 and 512 may be withdrawn at an appropriate time.

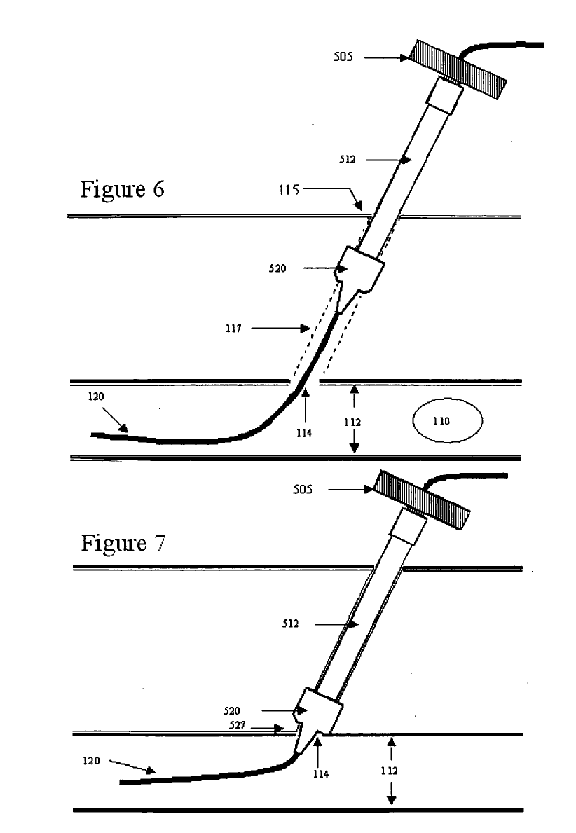

Referring back to FIG. 5A, the outer support tube 512 has a central lumen that houses the inner support tube 510. The inner support tube 510, in conjunction with the outer support tube 512, forms a stem that positions the plug 520 at the vascular puncture site 114 as further discussed below in reference to FIGS. 6 and 7. In some embodiments, the inner support tube 510 is made from stainless steel or some other material (such as another rigid metal or non-metal material).

Located concentrically within the inner support tube lumen is the central guidewire passageway 508, which extends through the tapered tip plug 520. This passageway 508 allows the guidewire 120 to be threaded through the sealing instrument 500, in order to facilitate the delivery of the plug 520 and the tubes 510 and 512 into the access tract 117 as further discussed below in reference to FIGS. 6 and 7.

FIG. 5A illustrates that the opposing end of the inner support tube 510 in some embodiments, extends beyond the outer support tube 512 and connects to a disc-shaped surface 505. As shown in FIGS. 5C, 6 and 7, the disc-shaped surface 505 is outside of the access tract 117 when the plug 520 is pushed into the access tract 117 and eventually placed at the vascular puncture 114. The operator can thus use the disc 505 to push the plug's tip 527 into the access tract 117 and rotate the tube 510, if necessary. The central passageway 508 extends through the disc shaped surface 505 in order to allow the sealing mechanism 500 to be pushed into the access tract 117 by threading the guidewire 120 through the plug 520, the inner support 510, and the disc-shaped surface 505. The surface 505 has other shapes in other embodiments.

In some embodiments, the outer support tube 512 is threaded to receive a threaded collar. Screwing the collar onto the tube 512 locks the inner tube 510 in a position within the outer tube 512. In this position, the inner tube 510 places pressure on the outer tube 512, which causes the outer tube's distal end to abut the interior surface of the cup 528 of the plug 520. The lateral pressure of the outer support tube 512 wall against the interior surface of the cup 528 is sufficient to allow the cup 528 to affix to and cooperate with the outer support tube 512 while the plug 520 is being positioned within the access tract 117.

To remove the inner tube 510 from its position within the interior of the outer support tube 512, the threaded collar is unscrewed from the tube 512. This releases the pressure that holds the inner support tube 510 within the outer support tube 512. Accordingly, the inner support tube 510 can be pulled out of its position within the interior of the outer support tube 512, so that the delivery mechanism formed by the tubes 510 and 512 can detach from the plug 520.

Other embodiments might couple the tubes 510 and 512 differently. Also, some embodiments might couple and decouple the tubes 510 and 512 and the plug 520 differently. For instance, in some embodiments, the inner tube 510 is movably placed within the outer support tube 512 such that it can move longitudinally within the outer tube 512. Other than a small amount of longitudinal motion, the inner tube 510 is locked in place within the outer tube 512. After the plug 520 is positioned within the puncture tract 117, the plug 520 may be detached from the tubes 510 and 512 by a clicking motion of the inner tube 510, whereby the inner tube 510 is quickly thrust downwards, towards the base of the plug's cup 528, and then released. As the inner tube 510 is released, the plug 520 detaches from the tubes 510 and 512.

b) The Plug

As illustrated in FIGS. 5A, 5C, 6 and 7, the inner and outer tubes 510 and 512 are used to place the plug 520 at the vascular puncture 114. When placed at the vascular puncture 114, the plug 520 occludes both the vascular puncture 114 and the access tract 117. The plug 520 then seals both the vascular puncture 114 and the access tract 117 and provides hemostasis to both.

The plug 520 has different shapes in different embodiments. FIGS. 5B and 5E illustrate the shape of the plug 520 in some embodiments. As shown in FIG. 5B, this plug 520 has a shoulder 526, a tapered tip 527, and a cup 528. The shoulder area 526 is designed to engage the perimeter of the vascular puncture 114. The tip 527 is designed to intrude into the vascular puncture 114 when the plug 520 is at the vascular puncture site 114. The cup 528 has an interior surface 525 that forms housing for receiving the tubes 510 and 512, as shown in FIG. 5A. As shown in FIGS. 5A and 5D, the cup 528 also includes a lumen 524, which defines the central passageway through the tip 527 and the cup section 528 of the plug 520. FIG. 5E illustrates some of the other possible shapes of the plug 520. Many of these shapes share the same attributes as the plug 520 in FIG. 5B.

In some embodiments, the plug 520 is bioabsorbable. In other words, in these embodiments, the plug 520 is made of bioabsorbable material or materials formulated to decompose and absorb into the organism at pre-determined rates, while promoting hemostasis. This has the beneficial effect of negating the need to open the tract post-operatively to extract foreign materials.

In some embodiments, the bioabsorbable plug 520 will typically be fashioned from a polymer with known physical characteristics and an expected bioabsorbability period of weeks to months. In other embodiments, much more rapidly absorbed compounds absorb or dissolve within hours to days of insertion in a patient. Examples of bioabsorable materials include glucose or related molecules. In some embodiments, the bioabsorbable polymer plug 520 is created in an injection molding process that precisely defines the shape and size of the plug 520.

Various materials used in or on the plug 520 also facilitate hemostasis. In some embodiments, the plug 520 is coated with, contains or is completely composed of Chitosan or other pro-coagulant material. In some embodiments, only a portion of the plug 520, for instance, its tip 527 and/or shoulder 526, is coated with, contains or is completely composed of Chitosan or other pro-coagulant material. In some embodiments, the coagulant will have a mixture of different materials, with different rates of coagulation, to allow a more controlled rate of coagulation and hemostasis. In some embodiments, a dissolvable coating or veneer of sugar, candy or a related polymer or crystal lies over the plug 520 to allow placement of the plug 520 prior to exposure of the procoagulant components to blood.

As illustrated in FIGS. 6 through 7, the outer support tube 512 and the inner support tube 510 (not shown) create a delivery mechanism to place a plug 520 precisely subcutaneously to intrude into and occlude the vascular puncture 114. In these embodiments, the outer support tube 512 forms the exterior wall for the sealing instrument 500. The inner support tube 510 is located concentrically within the outer support tube 512. Referring now also to FIGS. 5A, 14 and 15 (described in detail below), the distal end of each tube 510 and 512 rests firmly within, and cooperates with, the cup 528 of the plug 520. In this position, the tapered walls of the outer support tube 512 push laterally and firmly against the inner walls of the cup 528 of the plug 520.

To occlude the vascular puncture 114, the operator first threads the guide wire 120 into the central lumen 524 (which is defined through the plug, 520, the inner and outer support tubes 510 and 512, and the disc-shaped surface 505, as illustrated in FIG. 5A) until the plug's tip 527 is positioned at the vascular puncture 114. This insertion of the guide wire 120 is illustrated in FIG. 6. Using the marks 530 (illustrated in FIG. 5C) of the outer support tube 512 as a guide, the operator next advances the sealing mechanism 500 over the guide wire 120 to the distance into the access tract 117 that was measured in some embodiments using the measuring instrument 300.

Some users might not use the measuring device 300 and the marks 530 on the outer support tube 512 to position the plug 520 at the vascular puncture 114. Instead, the operator might simply move the plug 520 through the guide wire 120 and forward it into the access tract 117 until the shoulder 526 of the plug 520 meets firm resistance from the perimeter of the vascular puncture 114. When the operator feels this resistance, the operator stops pushing the plug 520 further into the access tract 117. When the plug's shoulder 526 rests on the outer surface of the blood vessel 110, the plug tip 527 occludes the vascular puncture 114. In some embodiments the entry of the plug's tapered tip 527 into the vascular puncture 114 will be signaled by bleeding visible at the proximal hub 310.

With the plug 520 in the vascular puncture 114, the blood flow stops. At this point, the patient is almost ready to be moved to a recovery area. In some embodiments of the mechanism 500, before being moved it is helpful to immobilize the sealing mechanism 500. One apparatus to do so is a clamping mechanism to secure the sealing mechanism 500 in position.

3. The Clamping Mechanism and the Removal of the Sealing Mechanism

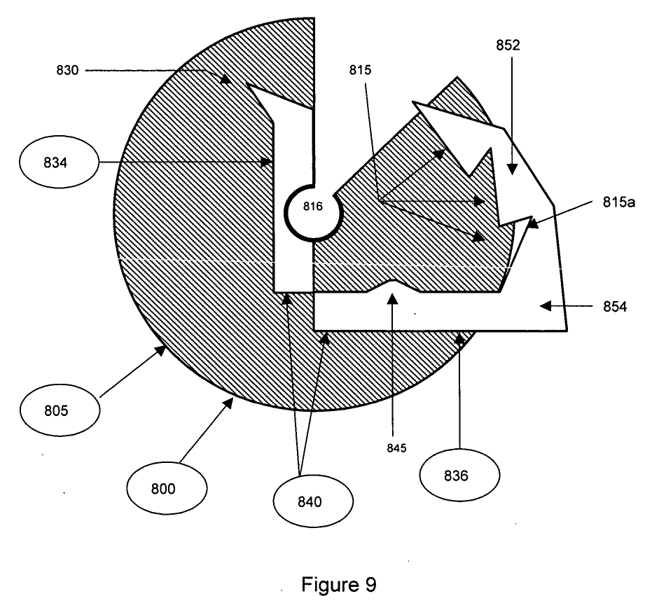

FIGS. 8 and 9 depict the clamp mechanism 800 of some embodiments of the invention. FIG. 8 is a perspective of the clamping mechanism 800. FIG. 9 is a top elevation of the clamp mechanism 800.

As illustrated these figures, the clamp mechanism 800 includes a surface plate 805, a rigid arm 834 and a moveable arm 836. As further described below, the rigid arm 834 and the moveable arm 836 combine to form a clamp 840 to immobilize the outer support tube 512 (shown in FIG. 8) during patient recovery. The surface plate 805 has a central lumen 816 and two opposing surfaces aligned along a latitudinal axis. One of the two latitudinal surfaces is located distally to the operator. The distal surface includes an adhesive surface 807 and a removable cover 808 (shown in FIG. 8) that protects the adhesive surface 807 until it is used. In some embodiments, the plate 805 includes a wedge shape opening, starting at the central lumen 816 and widening until it reaches the plate's perimeter. The wedge allows the clamping mechanism 800 to be placed around the outer support tube 512.

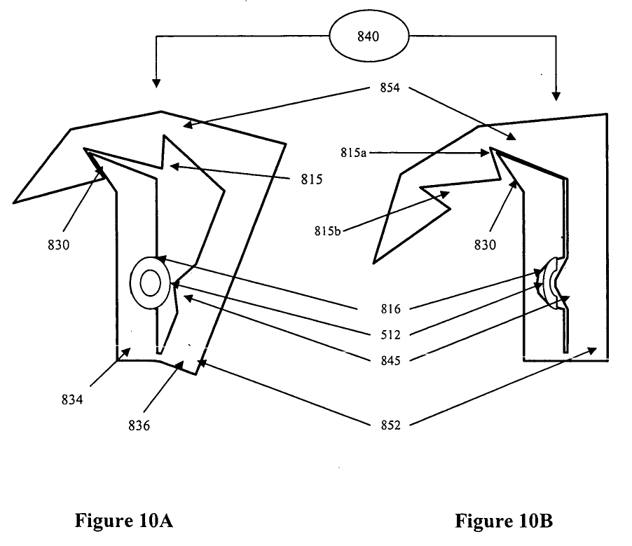

The rigid arm 834 fixedly attaches to the surface plate 805. As seen in FIGS. 10A and 10B, the central lumen 816 is also defined within the rigid arm 834. The rigid arm 834 also includes a tip 830 that locks the moveable arm 836 in one of two closed positions, as further described below.

As seen in FIGS. 8 and 9, the moveable arm 836 includes a first segment 852 and a second segment 854. The second segment 854 includes a protrusion 845 that is designed to enter the central lumen 816 and pin the outer support tube 512. The first segment 852 includes a series of notches 815. The notches 815 are shaped to receive and cooperate with the rigid arm tip 830 to allow the clamp 840 to form an open, partially closed or closed position. The open, partially closed or closed positions are defined by the position of the protrusion 845 through central lumen 816.

FIG. 9 illustrates the open position, FIG. 10A illustrates the partially closed position, and FIG. 10B illustrates the fully closed position. As shown in FIGS. 9 and 10A, when arms 834 and 836 are in open or partially closed positions, the protrusion 845 of movable arm 836 is positioned away from the central lumen 816, through which the outer support tube 512 goes through. In the open position, both the outer support tube 512 and the inner support tube 510 (not shown) are free to move. In the partially closed position, the inner support tube 510 is free to move and the outer support tube 512 is held by the clamp 840. Accordingly, the inner support tube 510 can be removed after placing the clamp 840 in the partially closed position. In other words, the partially closed position allows the clamp 840 to grasp the outer support tube 512 firmly without deforming or collapsing the outer support tube 512, thereby allowing the inner support tube 510 to be removed.

As illustrated in FIG. 10B, the fully closed position is defined by the protrusion 845 at least partially entering into the central lumen 816 and pinning into the outer support tube 512. The fully closed position firmly pinches the outer support tube 512 shut to prevent the flow of blood or fluid through the tube 512. As depicted in FIG. 10B, in the closed position, the clamp 840 is fully affixed to the outer support tube 512 and the outer support tube 512 is immobile. The operation of the clamp mechanism 800 will now be described. The operator applies the plate 805 to the outer support tube 512 by sliding the wedge and central lumen 816 of the plate 805 around or over the outer support tube 512, sliding the clamp mechanism 800 along the outer support tube 512 towards the epidermis 116 and affixing the adhesive surface 807 to the epidermis 116. To do so, the operator first removes the adhesive cover 808 of the plate 805 to expose the adhesive surface 807. The operator then affixes the clamp mechanism 800 to a surface (such as to the epidermis 116 of the patient) by attaching the surface plate 805 to the surface.

With the surface plate 805 in place, the operator moves the moveable arm 836 to the outer support tube 512 as illustrated in FIGS. 8, 10B, 12 and 13. The operator then moves the moveable arm 836 to a partially closed position (i.e. the tip 830 is engaged with the notch 815a) to secure the outer support tube 512. At this stage, the guidewire 120 may be withdrawn, as illustrated in FIG. 13.

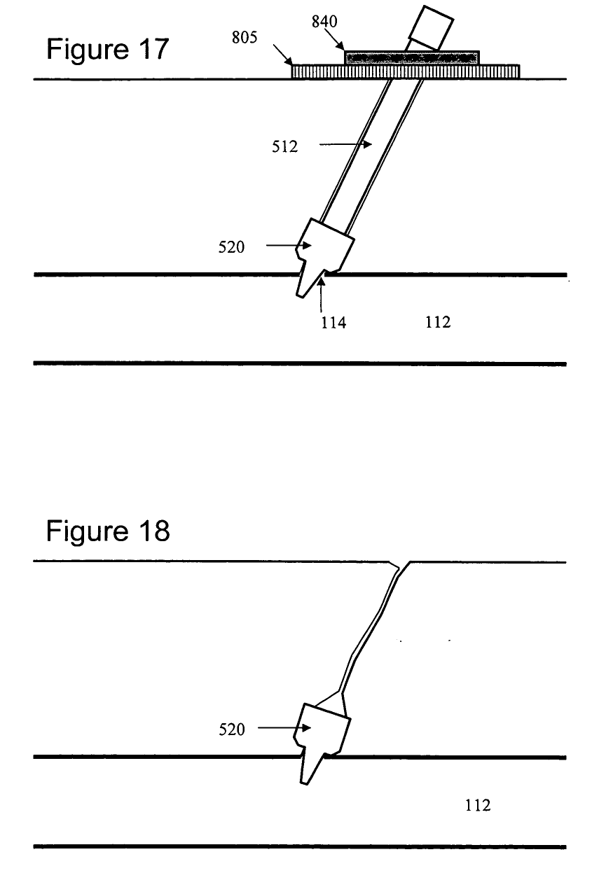

Once the sealing mechanism 500 is secured and the guidewire 120 is removed, the operator removes the inner support tube 510. The operator then moves the moveable arm 836 to a second and fully closed position wherein the tip 830 is engaged with the notch 815b (shown in FIG. 10B) to crimp the outer support tube 512, as shown in FIG. 17. In this position, the support tube 512 is held firmly in place and blood is prevented from flowing out the support tube 512.

With the outer support tube 512 secured, the patient may be remanded to a holding/recovery area with the surface plate 805 and clamp 840 in place as shown in FIG. 17. Once the patient has arrived at the post-surgery location and the healing has proceeded far enough, the outer support tube 512 and remaining apparatus are removed, as seen in FIG. 18. As shown in this figure, this leaves the plug 520 in place to assure complete hemostasis. As illustrated in FIG. 18, the withdrawal of the foreign objects causes the access tract 117 to close naturally and facilitates hemostasis within the tract 117. The plug 520 will dissolve and be harmlessly absorbed into the system.

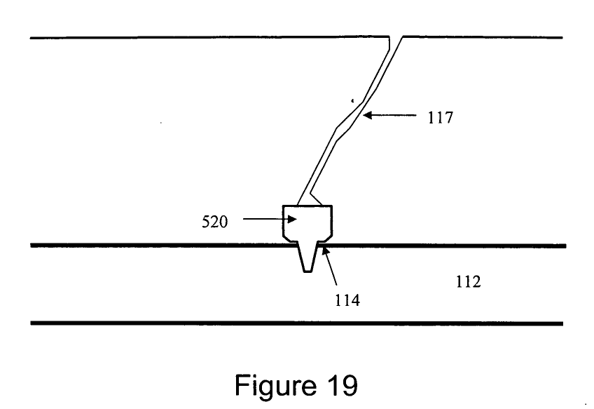

In the above-described embodiment, the clamping mechanism 800 is designed to immobilize the outer support tube 512 of the sealing mechanism 500. In other embodiments, the clamping mechanism 800 may also be used to immobilize a catheter or any other medical device that includes a stem extending outward beyond the epidermis 116. Additionally, in FIGS. 6-7, 11-13 and 17-18 the access tract is shown to be at an angle that is less than 90°. One of ordinary skill in the art will realize that the access tract 117 might be roughly perpendicular to the plane of the vascular puncture 114 in some cases, as shown in FIG. 19. This allows the tip 527 of the plug 520 to be positioned within the vascular puncture 114 while the shoulder 526 rests firmly on the periphery walls of the vascular puncture 114.

B. Method of Use

As mentioned above, the vascular puncture sealing mechanism is used to seal a vascular puncture and a vascular access tract upon conclusion of a medical procedure that creates a vascular puncture and an access tract. At the conclusion of an intravascular medical procedure, most of the instrumentation (e.g., all the instrumentation except the access sheath) used in the procedure is removed from the blood vessel and the access tract. A guidewire is re-inserted into the access tract (e.g., re-inserted through the access sheath). In some cases, the remaining instrumentation is removed.

Referring back to FIGS. 3 and 4, in some embodiments, the measuring mechanism 300 is threaded onto the guidewire 120 and into the access tract 117 to measure the distance from the epidermal layer 116 to the vascular puncture 114. In other embodiments, the measuring mechanism 300 is not used. As mentioned above, the measuring mechanism 300 is designed so that when its holes 316 enter the blood vessel 110, blood will appear at the proximal end of the measuring mechanism 300.

After the distal tip of the measuring mechanism 300 has entered the vascular lumen 112, the measuring mechanism 300 is slowly withdrawn (as shown in FIG. 4) from the vascular lumen 112. The withdrawal continues until the holes 316 exit from the vascular lumen 112 and enter the access tract 117. This entry is signaled by an abrupt drop in amount of blood at the hub 310. At this point, the operator may use the measurement indicators placed on the outside of the measuring mechanism 300 to determine the distance from the percutaneous opening 115 to the vascular puncture 114.

Referring next to FIGS. 5 through 7, having measured the distance from the epidermal layer 116 to the vascular puncture 114, the vascular puncture 114 and access tract 117 can be sealed by inserting the plug 520 of the sealing instrument 500 into the access tract 117 at the depth measured by the measuring device 300. Specifically, the sealing instrument's plug 520 and tubes 510 and 512 are inserted into the access tract 117 by threading the guidewire 120 through the central guidewire passageway 508 of the sealing instrument 500 and sliding the plug 520 and tubes 510 and 512 over the guidewire 120 and into the access tract 117.

In those embodiments that use a measuring device 300, the marks 530 (as shown in FIG. 5C) of the outer support tube 512 are used as a guide by the operator to advance the sealing mechanism 500 over the guide wire 120 to the distance into the access tract 117 that was measured using the measuring instrument 300. The marks 530 allow the operator to precisely position the plug 520 to occlude the blood vessel puncture site 114. As mentioned above, an operator might not use the measuring device 300 and the marks 530 on the outer support tube 512 to position the plug 520 at the vascular puncture 114. Instead, the operator might simply move the plug 520 forward in the access tract 117 until the shoulder of the plug 520 meets firm resistance from the vascular puncture 114. In some embodiments the entry of the plug's tapered tip 527 into the vascular puncture 114 will be signaled by bleeding visible at the proximal hub 310.

Still referring to FIGS. 5-7, when the plug 520 is securely within the blood vessel 110, the blood flow stops and the patient is almost ready to be moved to a recovery area. Before being moved, the clamping mechanism 800 is used in some embodiments of the mechanism 500 to immobilize the sealing mechanism 500 and its plug 520 during the recovery and healing process to prevent re-opening the wound. In some other embodiments, the support tube 512 may be detached from the plug 520 immediately after hemostasis is secured. In these embodiments, there is no need to clamp the outer support tube 512.

As illustrated in FIG. 8, the clamping mechanism 800 has an adhesive cover 808, a surface plate 805 and a clamp component 840. To affix the clamping mechanism 800 of some embodiments to the catheter or tube 512, the operator first removes the adhesive backing of the mechanism 800 to expose the adhesive surface 807. He then slides the mechanism 800 around any objects obtruding the epidermis 116 and affixes the surface plate 805 to the epidermis 116 by way of the adhesive surface 807. The clamp component 840 of the clamping mechanism 800 is then moved to a first and partially closed position (shown in FIG. 10A) to secure the outer support tube 512 and allow the withdrawal of the guidewire 120, as shown in FIG. 13. With this accomplished, the operator can then remove the inner support tube 510 (for example, by using one of the coupling mechanisms described above) and move the clamp 840 to a second and fully closed position (shown in FIG. 10B) to crimp and completely secure the outer support tube 512, as shown in FIG. 17.

With the vascular sealing mechanism 500 secured, the patient may be remanded to a holding/recovery area with the clamp mechanism 800 in place as shown in FIG. 17. Once the patient has arrived at the post-surgery location and had sufficient time for recovery, the clamp surface plate 805 and the clamp 840 are removed as a unit with the outer support tube 512, by lifting the adhesive surface plate from the skin and withdrawing the apparatus from the patient.

As shown in FIG. 18, this leaves the plug 520 in place during recovery to assure complete hemostasis. Ultimately, foreign objects except the plug 520 are removed from the access tract 117 following the intravascular procedure. As illustrated in FIG. 18, the withdrawal of the foreign objects from the access tract 117 causes the access tract 117 to close naturally and facilitates hemostasis within the tract 117. When the plug 520 is bioabsorbable, it will dissolve and be harmlessly absorbed into the system. Also, in the embodiments where the plug 520 contains one or more coagulating agents, the coagulating agents speed up hemostasis.

C. Alternative Embodiments and Method of Use

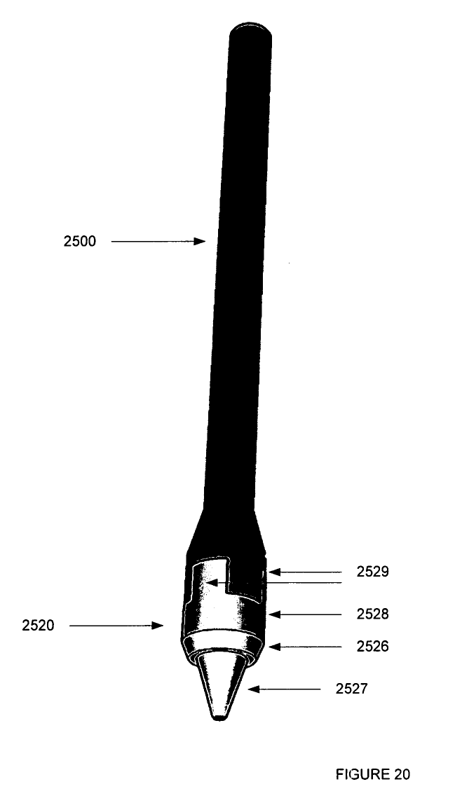

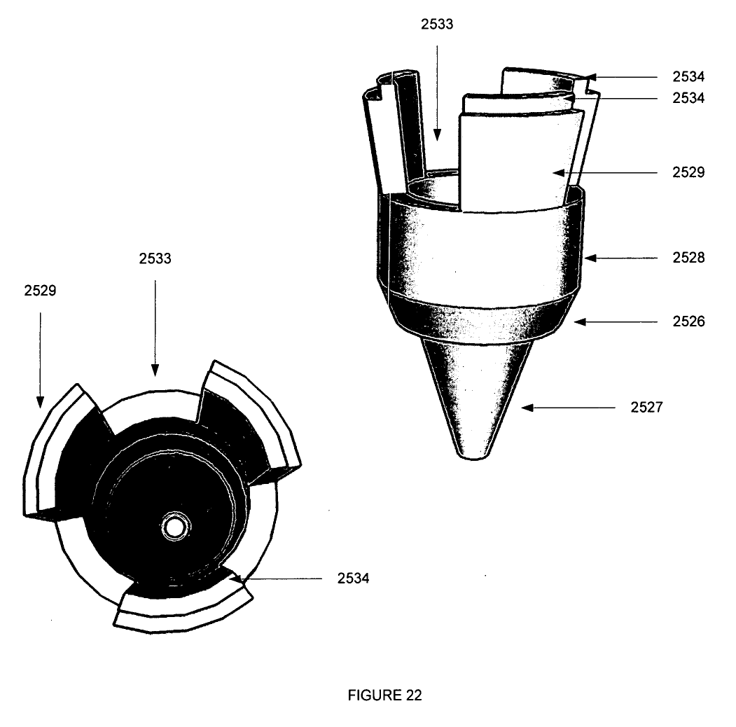

FIGS. 20-23 illustrate another plug 2520 and sealing mechanism 2500 of some embodiments of the invention. Like the plug 520 illustrated in FIGS. 5-7, the plug 2520 of the sealing mechanism 2500 has a shoulder 2526, a tapered tip 2527 (which is conical in this case) and a cup 2528 attached to an opposing end of the shoulder.

Also, like the sealing mechanism 500 illustrated in FIGS. 5-7, the sealing mechanism 2500 of some embodiments has an inner support tube 2510, an outer support tube 2512 and a central lumen defined longitudinally from the tip 2527 through the plug's shoulder 2526 and cup 2528. This central lumen is for passing a guidewire 120 through the sealing mechanism 2500. Also as before, the sealing mechanism 2500 has a proximal end that extends outward beyond the epidermis 116 and a distal side that extends into the access tract 117.

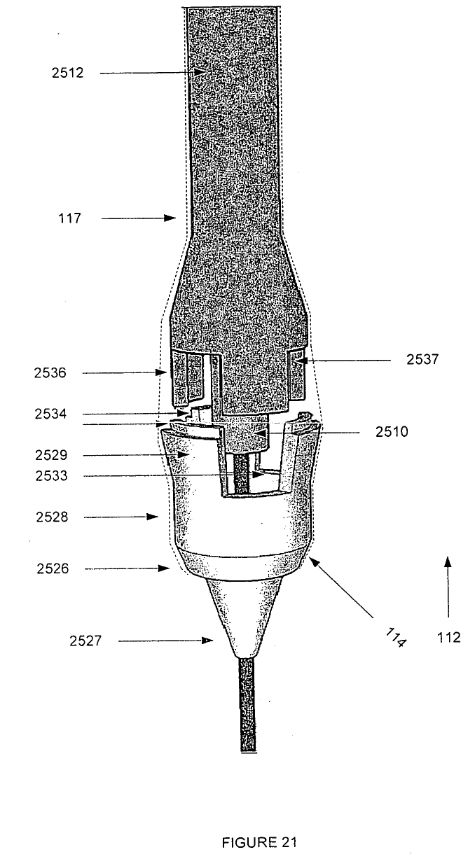

As illustrated in FIGS. 20 and 21, the plug 2520 has a proximal end that is detachably affixed to the distal end of the sealing mechanism 2500. The tip 2527 of the plug 2520 is at the plug's distal end. The tip 2527 is shaped to occlude and seal the vascular puncture 114 at any angle of entry into the vascular puncture 114. The plug's shoulder 2526 is attached to the tip 2527 on one end and to the cup 2528 at an opposing end. The shoulder 2526 is made of a semi-flexible material designed to occlude the vascular tissue surrounding the perimeter of the vascular puncture 114.

As seen in FIGS. 20-23, the plug 2520 has prongs 2529 that extend beyond the cup 2528. In some embodiments, a space between each adjacent prong 2529 defines a receptacle 2533 that is designed to cooperate with and receive a prong 2536 of the sealing mechanism 2500 (discussed below). In some embodiments, the prongs 2536 also include a lip or lips 2538 to cooperate with and receive a corresponding component of the sealing mechanism 2500 (discussed below).

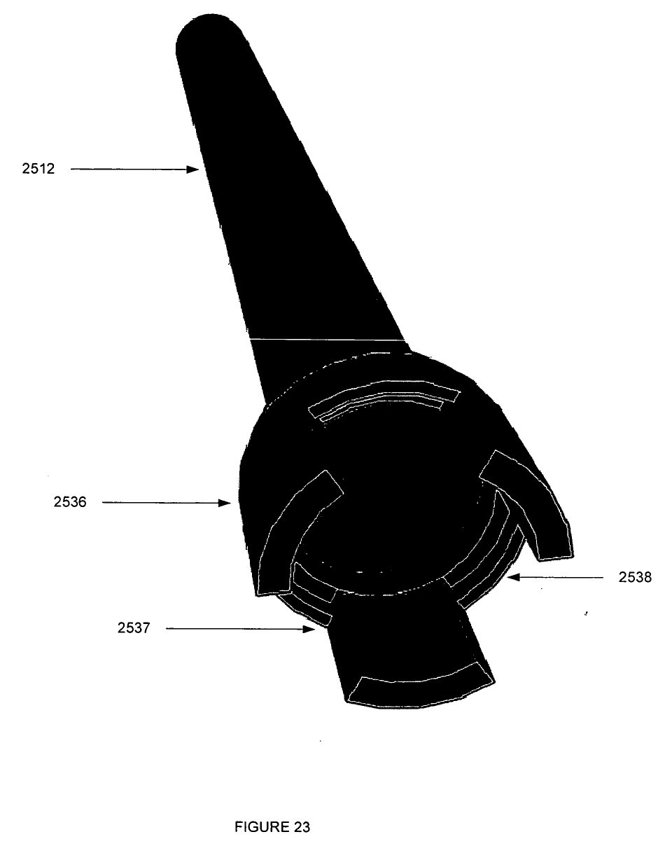

FIG. 21 illustrates tubes 2510 and 2512 and plug 2520. The tubes 2510 and 2512 functionally correspond to the previously discussed inner support tube 510 and outer support tube 512. However, the distal end of the outer support tube 2512 includes a flared section that extends radially outward at an angle. As illustrated in FIGS. 21 and 23, the flared section includes prongs 2536 and a receptacle 2537 placed between each pair of prongs 2536. Each receptacle 2537 has one or more lips 2538 that are designed to couple with the lips 2534 of the prongs 2529. As in sealing mechanism 500, the tubes 2510 and 2512 form a stem for the sealing mechanism 2500.

In some embodiments of the sealing mechanism 2500, the tubes 2510 and 2512 and the plug 2520 are joined prior to use. In some embodiments, such as shown in FIG. 21, the plug lips 2534 cooperate with the sealing mechanism lips 2538 to constrain the plug's prongs 2529 until the plug 2520 is detached from the sealing mechanism 2500.

In some embodiments, the inner tube 2510 is movably placed within the outer support tube 2512 such that it can move longitudinally within the tube 2512. Other than a small amount of longitudinal motion, the inner tube 2510 is locked in place within the outer tube 2512. After the plug 2520 is positioned within the puncture tract 117, the plug 2520 may be detached from the tubes by a clicking motion of the inner tube 2510, whereby the inner tube 2510 is quickly thrust downwards, towards the base of the plug's cup 2528, and then released. As the inner tube 2510 is released, the plug 2520 detaches from the tubes 2510 and 2512 and the prongs 2529 flare out to hold the plug 2520 at its current position. Other embodiments might use other techniques (e.g., some of the techniques described previously) to couple and de-couple (1) the tubes 2510 and 2512 and/or (2) the tubes 2510 and 2512 and the plug 2520.

The operation of the sealing mechanism 2500 will now be described. To position the plug 2520 so that it occludes and intrudes into the vascular puncture 114, the plug 2520 and the stem of the sealing mechanism 2500 are advanced over a guidewire 120 and into the access tract 117 until the plug 2520 meets resistance from the perimeter wall of the vascular puncture 114. Next, sufficient pressure is applied to ensure that the plug 2520 occludes and intrudes into the puncture 114. Hemostasis occurs almost immediately such that the remainder of the sealing mechanism 2500 may be quickly detached from the plug 2520 and removed.

When the plug 2520 separates from the sealing mechanism 2500, the prongs 2529 are released from their constraints and flare outward against the wall of the access tract 117. When so positioned, the friction between the flared out prongs 2529 of the plug 2520 and the interior of the access tract 117 cause the plug 2520 to resist movement in the access tract 117. This resistance, as well as pressure from the access tract 117 collapsing on itself (as depicted in FIG. 18), assists to keep the plug 2520 stationary within the access tract 117. As the plug 2520 is bioabsorbable, it dissolves and absorbs into the patient while the wound is healing, negating any need to re-open the wound to withdraw instrumentation. With the removal of the sealing mechanism 2500 and with the plug 2520 occluding the access tract 117, a bandage is applied to the epidermis 116.

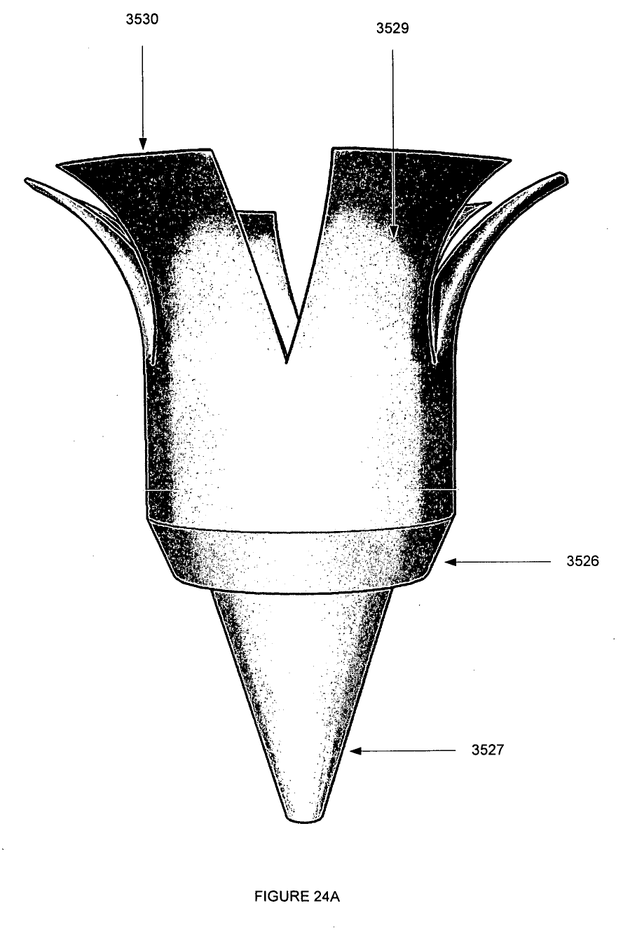

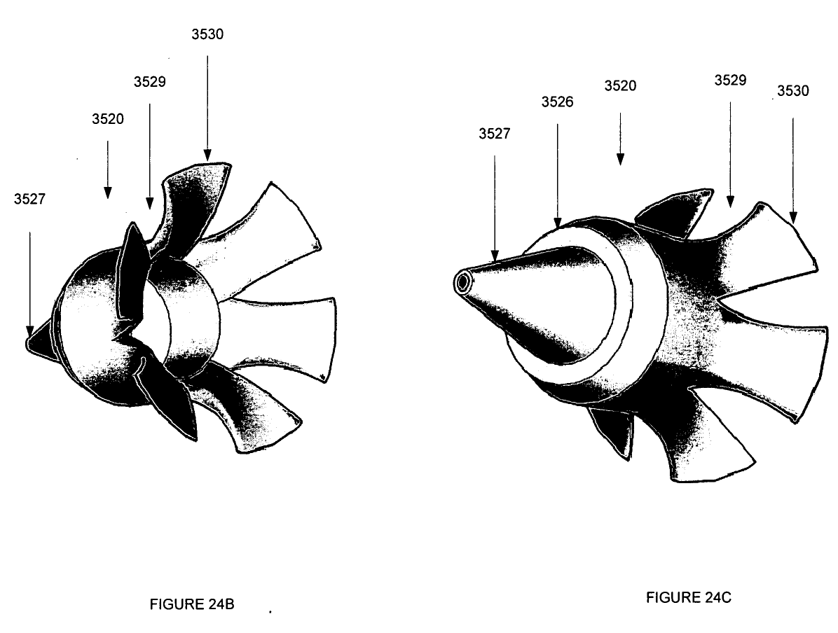

FIGS. 24A, 24B and 24C illustrate a plug 3520 of other embodiments of the invention. Like the plug 2520, the plug 3520 has prongs 3529. Plug 3520 also has a shoulder 3526, a tapered tip 3527, and prong ends 3530. Unlike the inline prongs 2529 of plug 2520, the prongs 3529 arch radially outwards at all times and are more flexible. In a confined environment, such as the puncture tract 117, movement into the tract 117 causes the prong ends 3530 of the prongs 3529 to flex inward to contract the circumference formed by the prong ends 3530.

As with the plug 2520, the plug 3520 is inserted by threading it and the delivery mechanism onto the guidewire 120 and advancing both into the puncture tract 117. Also like the plug 2520, the plug 3520 uses a delivery mechanism (not shown) to advance it in the tract 117 to intrude into and occlude the vascular puncture 114. While the plug 3520 is being advanced towards the vascular puncture 114, the interior wall of the access tract 117 causes the prongs 3529 to contract. This allows the prongs 3529 to glide against the walls of the puncture tract 117. When the plug 3520 intrudes into and occludes the puncture 114, the outwardly flaring prongs 3529 of the plug 3520 hold it in place. With the plug 3520 sealing the vascular puncture 114, the delivery mechanism is withdrawn and a bandage placed over the percutaneous opening 115. Hemostasis begins almost immediately.

In different embodiments, the plug 3520 couples to and de-couples from the delivery mechanism differently. For instance, in some embodiments, the plug 3520 couples to and de-couples from a delivery mechanism formed by the inner and outer support tubes (similar to tubes 510 and 512) through a clicking motion of the inner tube, as described above. Other embodiments couple differently. Moreover, in some embodiments, the plug 3520 and delivery mechanism are not coupled at all. Instead, after the delivery mechanism positions the plug 3520 within the puncture tract 117, the two are separated by pulling the delivery mechanism away from the plug 3520.

As will be appreciated from the foregoing, the invention and the method of use described herein enables the fast and effective sealing of a vascular puncture. This mechanism presents a significant advance in the fields of cardiology, radiology and vascular surgery. It significantly improves upon the prior art by providing an effective means of completely sealing a vascular access puncture site, even in anti-coagulated patients, without leaving potentially harmful materials within the instrumented vasculature or requiring the withdrawal of removable components through a hemostatic clot with the possibility of bleeding and hematoma formation. This innovation is expected to reduce patient discomfort, improve sheath related complication rates due to bleeding and hematoma formation, reduce intra-arterial trauma, reduce hospitalization time and allow rapid mobilization and earlier discharge of patients following catheter based vascular procedures.

While the invention has been described with reference to numerous specific details, one of ordinary skill in the art will recognize that the invention can be embodied in other specific forms not detailed herein without departing from the spirit of the invention. Some embodiments seal a vascular puncture site and access tract after an intravascular procedure. Other embodiments may be used without a prior intravascular procedure. In some embodiments, some parts of the apparatus form a single unit without affecting the utility or method of operation of the mechanism. Thus, one of ordinary skill in the art would understand that the invention is not to be limited by the illustrative details provided herein, but rather is to be defined by the appended claims.

What is claimed is:

1. An apparatus for achieving hemostasis in a vascular puncture and puncture tract that are created during a medical procedure on a patient, the apparatus comprising:
(a) a plug for placement within the vascular puncture and puncture tract to intrude into and to occlude the vascular puncture, and
(b) a delivery mechanism for delivering the plug into the puncture tract.

2. The apparatus of claim 1, wherein the delivery mechanism is removed from the puncture tract after hemostasis has been achieved.

3. The apparatus of claim 1, wherein the plug is bioabsorbable.

4. The apparatus of claim 1, wherein the plug includes a pro-coagulant material.

5. The apparatus of claim 1, wherein the plug is composed of Chitosan.

6. The apparatus of claim 1, wherein the plug is coated with Chitosan.

7. The apparatus of claim 1 further comprising a lumen that is defined through the plug and the delivery mechanism, said lumen for passing a wire through the plug and the delivery mechanism in order to guide the plug into the puncture tract.

8. The apparatus of claim 1 further comprising an affixing structure for affixing the delivery mechanism to the patient while the plug is within the puncture tract.

9. The apparatus of claim 8, wherein the affixing structure includes a clamping mechanism for affixing the delivery mechanism to the patient while the plug is within the puncture tract.

10. The apparatus of claim 8, wherein the affixing structure includes a pad having at least one adhesive surface to apply onto a patient while the plug is within the vascular puncture and puncture tract.

11. The apparatus of claim 1, wherein the plug has a tapered tip for passage through the access tract and insertion into the vascular puncture.

12. The method of claim 11, wherein the tapered tip includes a pro-coagulant material.

13. The method of claim 11, wherein the tapered tip is composed of Chitosan.

14. The method of claim 11, wherein the tapered tip is coated with Chitosan.

15. The apparatus of claim 11 wherein said delivery mechanism comprises a plurality of tubes, said plurality having a first tube comprising:
a) a deformable material; and
b) a sidewall having an inner surface and an outer surface, a proximal end and a distal end.

16. The apparatus of claim 12 wherein the sidewall tapers inwardly such that it is thinner at the end that is proximal to a percutaneous opening and thicker at the distal and opposing end.

17. The apparatus of claim 12 wherein the sidewall has uniform thickness and bends inwardly such that the circumference of the first tube is larger at one end and smaller at an opposing end.

18. The apparatus of claim 12 wherein said plurality of tubes further comprises at least a second tube, said second tube comprised of stainless steel or other metal and having a central lumen, wherein said second tube is seated within said first tube.

19. The apparatus of claim 15 wherein said second tube is longer than the first tube.

20. The apparatus of claim 15 wherein said second tube further comprises a sidewall having uniform thickness, a proximal and a distal end.

21. The apparatus of claim 17 wherein the distal end of said first and second tubes are seated within said plug for delivery.

22. The apparatus of claim 17 wherein said second tube flares at the proximal end into a disc shape handle, said handle used to withdraw said second tube within said first tube.

23. The apparatus of claim 12 wherein said delivery mechanism comprises a plurality of tubes, each tube except the last tube of said plurality affixed concentrically within another tube, said last tube having all remaining tubes of said plurality concentrically placed within said last tube.

24. A method of achieving hemostasis in a vascular puncture and puncture tract that is created during a medical procedure on a patient, the method comprising:
(a) inserting a plug within the puncture tract so as to occlude the vascular puncture, and
(b) maintaining the plug in the vascular puncture and puncture tract until hemostasis is achieved.

25. The method of claim 21, wherein the plug is bioabsorbable.

26. The method of claim 21, wherein the plug includes a pro-coagulant material.

27. The method of claim 21, wherein the plug is composed of Chitosan.

28. The method of claim 21, wherein the plug is coated with Chitosan.

29. The method of claim 21 further comprising passing a wire through a passageway that is defined in the plug in order to guide the plug into the puncture tract.

30. The method of claim 26, wherein inserting the plug comprises using a delivery mechanism, to which the plug is affixed, to insert the plug into the vascular puncture and puncture tract, wherein the method further comprises affixing the delivery mechanism to the patient while the plug is within the puncture tract.

31. The method of claim 21, wherein the plug has a tapered tip for passage through the access tract and insertion into the vascular puncture.

32. A method of performing a medical operation, the method comprising:
a) defining a puncture tract and vascular puncture to access a blood vessel in a patient;
b) maintaining the plug in the puncture tract and occluding the vascular puncture until hemostasis is achieved.
